(19) United States
(12) Patent Application Publication
(10) Pub. No.: US 2007/0190117 A1
(43) Pub. Date: Aug. 16, 2007

(54) Buccal formulations of galanthamine and uses thereof

(76) Inventors: Bodo Asmussen, Bendorf (DE); Joachim Moormann, Werne (DE)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(21) Appl. No.: 10/569,160

(22) PCT Filed: Apr. 23, 2004
(86) PCT No.: PCT/EP04/04325

§ 371 Date: Oct. 17, 2006

(30) Foreign Application Priority Data
Aug. 19, 2003 (DE) 103 38 544.4

(51) Int. Cl. A61K 9/70; A61K 31/55

(52) U.S. Cl. 424/443; 514/214.01

(57) ABSTRACT

Film-shaped medicaments for buccal administration of galanthamine or of a salt or derivative thereof comprise at least one layer which contains a cholinergic active agent acting on the central nervous system or a combination of at least two such active agents, the said substance(s) being selected from the group comprising galanthamine, pharmaceutically acceptable salts of galanthamine, galanthamine derivatives and their pharmaceutically acceptable salts.

The invention relates to film-shaped medicaments for buccal administration of galanthamine and of the salts and derivatives thereof, as well as the use of these medicaments for the treatment of diseases or symptoms of diseases.

Galanthamine (4a,5,9,11,12-hexahydro-3-methoxy-11-methyl[6H]-benzofuro-[3a,3,2ef][2]benzazepine-6-ol) is a brain-penetrant inhibitor of the cholinesterase enzymes and a modulator of neuronal nicotinic acetylcholine receptors (NACHRs). The latter are located on presynaptic endings of various nerve paths, especially of cholinergic and dopaminergic nerve paths, and can be activated either by the natural ligand acetylcholine or by synthetic ligands. In low concentrations, galanthamine acts on NACHRs in a direct way as a so-called allosterically potentiating ligand (APL) which increases the response of these receptors on acetylcholine. Since, in this concentration range, galanthamine simultaneously increases the concentration of acetylcholine in the synaptic region as inhibitor of the catabolic enzyme acetylcholinesterase, it shows a particularly pronounced increase of cholinergic neurotransmission.

This strong cholinergic action of galanthamine is made use of in the therapy of the central cholinergic deficit in Alzheimer's disease. The hydrobromide salt is approved, in the form of tablets which release the active substance immediately (WO 97/47304), and a drinking solution, both under the trade name of Reminyl, for the therapy of light to moderately severe Alzheimer's dementia.

Galanthamine and its salts are used or have been taken into consideration for the treatment of various further diseases and disease symptoms, which include:
paralytic states in or as a result of: poliomyelitis, myasthenia gravis, brain and spinal cord injuries (Göpel et al., Psychiat. Neurol. Med. Psych. 23, 712-718 (1971));
chronic fatigue syndrome (EP-B-0 584 185);
schizophrenia (EP-B-0 584 285);
sleep disturbance, especially snoring and apnoe (WO 97/22339);
the effects of jetlag (EP-B-0 764 025);
disorders of the central nervous system and intoxications caused by action of psychotropic substances (DE-A-101 19 862), poisonings by neurotoxins (DE-C-43 42 174);
alcoholism (DE-C-40 10 079) or nicotine dependence (DE-C-43 01 782; DE-A-101 34 038);
as antidote for neuroleptic analgesia (Cozanitis et al., J. Amer. Med. Assoc. 240, 108 (1978)).

Since galanthamine, as hydrobromide (or in the form of another pharmacologically acceptable salt) is on the one hand completely absorbed from the gastrointestinal tract, but on the other hand has a relatively short half life of approx. 5 h in plasma, a saw tooth-like time course of the galanthamine plasma concentration results when administering direct-release (i.e. without delay) dosage forms since, to achieve a scheme of two oral administrations per day, it is necessary to administer unnecessarily high doses in order to maintain the plasma concentration in the therapeutically effective range of approx. 10 to 25 ng/ml for as long a part of the time interval between dose administrations as possible. With this administration scheme it must be accepted that immediately after administration of the galanthamine preparation plasma concentrations of markedly above 40 ng/ml are achieved in an uncontrolled manner, which, in particular in patients not previously treated with galanthamine, may lead to peripheral, especially gastrointestinal and cardiovascular, side effects (intestinal cramps, diarrhoea, hypotension).

There have thus been various attempts at developing dosage forms with controlled, retarded release of galanthamine which achieve an approximately trapezoidal time course of the plasma concentration over a period of 24 to 48 h whereby the concentration plateau can be maintained for approx. 24 hours in a therapeutically effective range that is, however, still free from side effects for most patients. A tablet with delayed active substance release (described in WO-A-00 38 686) is currently in the approval stage for worldwide approval for the indication of Alzheimer's dementia.

As tablets, these dosage forms have the disadvantage of not being suitable for patients with difficulty in swallowing and that they need to be taken with liquid. On the other hand, there has also been developed a transdermal therapeutic system (TTS) which contains galanthamine in the form of its free base (DE-C-43 01 783, DE-C-40 10 079) and which has been clinically tested especially with regard to its use for the therapy of alcohol abuse (DE-C-40 10 079). Since the plasma concentrations which are optimal for the treatment of the craving for alcohol are similar to those required in the therapy of dementia, such galanthamine-containing TTSs could also be utilized in the therapy of Alzheimer's disease. In formulations with direct release, maximum plasma concentrations of galanthamine are achieved after 30 to 60 min, in the case of be above-described delayed release formulations this occurs after several hours.

However, in particular on account of the nature of addiction behaviour, with substance cravings that recur again and again—often following long-lasting abstinence—and are difficult control, such dosage forms also appear desirable as bring about an onset of action of galanthamine which is attainable within a few minutes. In the treatment of other diseases or symptoms, too, a quick onset of action may be desirable. This is, however, not the case with the TTSs described above.

The object of the present invention was therefore to provide dosage forms for administration of galanthamine (or of a salt or derivative thereof for treating diseases or disease symptoms which are accompanied, or caused, by a lack of acetylcholine-induced conduction and/or by disturbed regulation of neuronal nicotinic receptors, and which dosage forms are, on the one hand, to afford a rapid onset of action without the occurrence of unacceptable peripheral side effects and, on the other hand, are to avoid the above mentioned disadvantages of known dosage forms, especially of tablets.

Surprisingly, it has been found that these objects are achieved by film-shaped medicaments for buccal administration according to claim 1 and according to the claims dependent on said claim, as well as by the use of such medicaments for the treatment of the diseases and symptoms mentioned in claims 13 to 24.

In light of the above, it was absolutely to be expected that the use of a rapidly releasing formulation where the onset of action occurs within a few minutes following application would have to involve considerable side effects. Surprisingly, it turned out that it was possible to configure a buccal dosage form in such a way that the active substance shows the desired effects on the central nervous system within a short time, but without unacceptable peripheral side effects having to be accepted. This is achieved by providing a formulation of the medicament in the form of a film-shaped medicament for buccal administration. The term “buccal administration” is understood to mean that the medicament releases the active substance(s) in the region of the oral cavity, so that the active substance(s) can be absorbed via the oral mucosa (i.e. transmucosal absorption). This leads to a quick onset of action during the application period the wafer absorbs saliva and the active substance is released from the wafer to the outside into the oral cavity and absorbed via the oral mucosa. In the contact region of the application area, the active substance can be delivered directly from the wafer to the underlying mucosa. The onset of the active substance release occurs already after a very short lag period (approx. 10 s to 5 min) after starting the application.

Especially suitable are medicaments of the above-mentioned type which contain a freely water-soluble galanthamine salt (or a freely water-soluble salt of a galanthamine derivative) in a biocompatible matrix (as active substance reservoir), which matrix is introduced into the oral cavity for application. With preference, the said matrix is soluble in saliva.

The formulations according to the invention have the additional advantage of the patient being able to administer them to himself readily at any time, that is, even when no liquid is available, or when the patient suffers from difficulty in swallowing. In addition, the medicament can be taken in a more inconspicuous manner as compared to tablets, for example, since no liquid is necessary; this increases the patient's willingness to take the medicament considerably. Also, the application of the film-shaped medicament to the oral mucosa is not felt to be unpleasant by the person treated on account of the medicament's small thickness.

Moreover, the inventive medicaments can be used to advantage for medicament therapy in veterinary medicine, especially as mucoadhesive dosage forms.

The inventive film-shaped medicaments are flat dosage forms, preferably in the form of thin lamellas or wafer-shaped objects (also called “wafers”), which preferably have a total layer thickness in the range from 0.01 to 5 mm, with particular preference in the range from 0.03 to 2 mm, especially in the range from 0.05 to 1 mm. The film-like medicaments are applied orally and are preferably equipped with mucoadhesive properties in order to enable them to adhere to the oral mucosa (especially buccal or sublingual application, or in the area of the gums or the palate).

The wafer may be present as a dense object, the density preferably being between 0.3 g/cm³ and 1.7 g/cm³, with particular preference between 0.5 g/cm³ and 1.5 g/cm³, especially between 0.7 g/cm³ and 1.3 g/cm³. Advantageously, the flat body of the individual wafers may be round, oval, triangular to quadrangular or polygonal, or be of any desired geometric shape.

The invention furthermore comprises embodiments where at least one side or surface of the wafer, or both sides, is/are provided with a plurality of elevated structures or/and recessions, for example knobs, ribs or grooves.

Preferably, the inventive medicaments contain the active agent galanthamine in the form of one of its water-soluble, pharmaceutically acceptable salts, or in the form of a complex salt, with galanthamine hydrobromide being especially preferred. However, galanthamine may also be contained in the medicaments in the form of its free base. Also considered as active substances are galanthamine derivatives having an effect similar to that of galanthamine—or possibly a stronger or weaker effect—provided that they are able to pass through the blood-brain barrier and do not cause unacceptable side effects; also suitable are the pharmaceutically acceptable salts of such derivatives.

Suitable galanthamine derivatives and salts thereof have been described, for example, in WO-A-01 74820, EP-B-0 854 873, EP-B 0 853 624, EP-B-0 653 427, EP-B-0 648 771, EP-B-0 649 846 or in U.S. Pat. Nos. 5,958,903, 6,093,815, 6,150,354, 6,268,358, 6,319,91.

Galanthamine can be isolated from the bulbs of galanthus species, for instance by the process described in EP-B-0 815 112; alternatively, galanthamine can also be produced synthetically (e.g. Shimizu et al.; Heterocycles 8, 277-282 (1977)).

The content of the afore-mentioned publications is part of the disclosure of the present invention.

The invention comprises both the use of racemic mixtures of the active substances mentioned and of enriched or isolated enantiomers.

The medicaments according to the invention may optionally contain a combination of two or more of the aforementioned active ingredients. The total active substance content, relative to the active substance-containing layer(s), preferably amounts to 0.1 to 30%-wt, with particular preference 1 to 20%-wt, especially 5 to 15%-wt.

The active substance dose contained is preferably in the range from 1 to 500 mg, especially 10 to 100 mg.

Optionally, the inventive wafers may in addition contain one further active substance from the group of acetylcholinesterase inhibitors which is not selected from the group of galanthamine and its derivatives. Furthermore, the inventive wafers may additionally contain at least one active substance which is not selected from the group of the acetylcholinesterase inhibitors; thus, wafers employed in the treatment of nicotine abuse may in addition contain opiate antagonists, for example.

The structure of the film-like medicaments contains at least one layer. This layer, or at least one of several layers, preferably has a polymer matrix which serves as an active substance reservoir. The polymer content is preferably 5 to 95%, preferably 15 to 75%-wt, with particular preference 20 to 50%-wt, relative to the respective layer.

Polymers preferred for the production of the polymer matrix are, in particular: cellulose ether, especially ethyl cellulose, hydroxyethyl cellulose, propyl cellulose, carboxymethyl cellulose, Na-carboxymethyl cellulose, hydroxypropyl cellulose, hydroxypropylmethyl cellulose, hydroxypropylethyl cellulose, mixtures of cellulose ethers, cellulose acetate, polyvinyl alcohols (fully or partially hydrolysed), polyvinyl acetate, polyvinyl pyrrolidone, polyethylene oxide polymers, polyethylene glycols, polyurethane, polyacrylic acid, polyacrylate, polymethacrylate; poly(methyl vinyl ether-maleic acid anhydride; e.g. Gantrez® types such as Gantrez-AN, -S, -ES, -MS, especially Gantrez® AN 119, Gantrez® AN 117, Gantrez® MS 955 (by ISP); alginates, pectins, gelatine; polysaccharides, especially starch and starch derivatives, e.g. tapioca starch; natural gums.

The afore-mentioned components may also be employed in combination or as a mixture containing two or more of the components.

According to a preferred embodiment of the invention, the film-shaped medicament has the property of being soluble in aqueous media and/or of quickly disintegrating in aqueous media, but it is not mucoadhesive.

The term physiological media is, in particular, understood to mean water and physiological liquids such as saliva and mucus.

Rapidly disintegrating films are understood to be those films which completely, or essentially completely, disintegrate in an aqueous medium within 2 min, preferably 60 s, with particular preference 10 s, at a temperature of 37° C.

According to a further preferred embodiment, the film-shaped medicaments have mucoadhesive properties to enable them to stick to the oral mucosa during the period of application, and under the aforementioned conditions they are insoluble or not disintegratable, or only partially soluble or disintegratable, in aqueous media. “Mucoadhesive” means that at least one side of the surface of the film-like medicament is mucoadhesive; “only partially soluble or disintegratable” means that during the period of application (approx. 2 h to 24 h) less than 50%-wt, preferably less than 70%-wt, especially less than 90%-wt of the preparation (leaving the amount of active substance released out of consideration) is present in non-dissolved or non-disintegrated state.

According to a further preferred embodiment, the film-shaped preparations are characterized by either being mucoadhesive and soluble or disintegratable in aqueous media, or by being mucoadhesive and capable of gelling or swelling in aqueous media.

The disintegration time is preferably 10 s to 12 h, with particular preference 1 min to 1 h, especially 3 min to 15 min.

The mucoadhesive properties as well as the disintegration and solubility properties are mainly determined by the type(s) of polymer(s) forming the matrix, as well as by the relative portions of these polymers.

The following polymers are considered, with preference, as matrix-forming polymers which may be components of an inventive mucoadhesive formulation (without excluding other suitable raw materials known to those skilled in the art); these polymers can be utilized singly or in different combinations: polyvinyl alcohols (e.g. Mowiol®), cellulose derivatives such as hydroxypropyl methyl cellulose, hydroxypropyl cellulose, sodium carboxymethyl cellulose (e.g. Walocel®), methyl cellulose, hydroxyethyl cellulose and hydroxypropyl ethyl cellulose; starch and starch derivatives; gelatine (various types); polyvinyl pyrrolidone; gum arabic and other gums; pullulan; acrylates; polyacrylamide.

Polymers suitable as water-soluble (or disintegratable) or/and swellable or/and gel-forming polymers are, in particular, those from the following group: starch and starch derivatives, dextran; cellulose derivatives (as described above; as well as carboxymethyl cellulose, ethyl cellulose, propyl cellulose); polyvinyl alcohols, polyvinyl acetate, polyacrylic acid, polyacrylates, polyvinyl pyrrolidone, polyethylene oxide polymers, polyacrylamides, polyethylene glycol, gelatine, collagen and other gel-forming proteins; alginates, pectins, pullulan, xanthan, tragacanth, chitosan, alginic acid, arabinogalactan, galactomannan, agar-agar, agarose, carrageenan, natural gums. The aforementioned substances may be employed singly or in different combinations.

The film-forming medicaments according to the invention may furthermore be produced in the form of solidified foams. This embodiment is preferred, in particular, for film-shaped preparations which rapidly disintegrate in aqueous media. On account of their large interior surface and their relatively stiff configuration, they are characterized, on the one hand, by an excellent “mouthfeel” and, on the other hand, they enable a particularly quick active substance release. The density of these dry foams preferably lies between 0.01 g/cm³ and 0.9 g/cm³, with particular preference between 0.08 g/cm³ and 0.4 g/cm³, especially between 0.1 g/cm³ and 0.3 g/cm³. The volume used for calculating the thickness is defined by the volume filled by the total body of the wafer. Such foam-like wafers can be manufactured by the process described in DE-A-100 32 456, for example.

The manufacture of the inventive film-shaped medicaments is generally accomplished by initially producing a coating mass, which contains matrix polymer(s), active substance(s) and possibly auxiliaries in a solvent or solvent mixture, and by coating this mass onto an inert support to give a moist film, e.g. by doctor-knife, roller application, spraying or extrusion processes. This film is dried (or allowed to solidify) and separated, according to requirements, into dosage units of desired surface area (and with a defined active ingredient content). Preferably, the dried film is separated into surface sections of a size of less than 10 cm², with particular preference less than 8 cm², and especially less than 4 cm². The manufacture from a melt containing the above-mentioned components is also considered.

According to a preferred embodiment, the inventive wafers are characterized in that they release the active substance(s) into the oral cavity within 30 min, preferably within 15 min, with particular preference within 5 min, following application so that an effective plasma level is obtained.

Furthermore, it is provided for the film-shaped medicament to optionally have a bilayer or multilayer structure, with at least one of the layers containing active substance. The individual layers may differ from each other in terms of one or more of the following parameters: polymer composition, type of active substance, active substance concentration, solubility or disintegration properties, swelling capability, mucoadhesive properties, content of auxiliaries. Multilayer films can, for instance, be obtained by initially preparing a first film layer (as described) and, after drying, applying a further layer on top of this layer. As an alternative, two or more layers may be manufactured in separate process steps and these layers are then laminated to one another.

According to a particularly preferred embodiment, the active substance-containing layer of the wafers, or at least one of the layers, has a delayed time course of active substance release. This enables an active substance release over a period of preferably up to 6 h, with particular preference up to 12 h, and most preferably up to 24 h. The retardation of the release can be achieved by measures known to those skilled in the art, for example by determining the composition (polymers, auxiliaries), density and water insolubility of the respective matrix layer, by providing a control membrane or by encapsulating the active substance in polymer particles. By means of the above-described measures it is possible to control the time course of the active substance release in numerous ways.

In the case of multilayer film-shaped preparations it is preferred that these have a mucoadhesive layer which is preferably water-soluble or disintegratable and which contains the active substance(s). This layer is followed, in distal direction (i.e. towards the oral cavity), by at least one further layer which preferably exhibit(s) a retarded active substance release. In this way it is made possible to achieve a quick onset of action on the one hand and, on the other hand, the release of a maintenance dose over an extended period of time.

Furthermore, one may make use of the measure of providing at least one of the distal layers as a layer which is soluble or disintegratable in aqueous media in order to ensure a quick initial onset of action. Also, one of the layers, preferably one of the distal layers, especially the outermost layer, may be configured as a barrier layer in order to slow down or prevent the diffusion of water and/or active substance. This barrier is insoluble or only slowly soluble in aqueous media and is preferably free of active substance.

The inventive formulations may additionally contain one or more auxiliaries, especially auxiliaries from the following groups:

Fillers (e.g. SiO₂, titanium dioxide, zinc oxide, chalk, activated charcoal, maize starch); colourants; emulsifiers (e.g. polyethoxylated sorbitan fatty acid esters, polyethoxylated fatty alcohols, lecithin); plasticizers (e.g. polyethylene glycol, glycerol, sorbitol, mannitol and other sugar alcohols, dexpanthenol; polyalcohols such as glycerol, propanediol, butanediol, mygliol; higher alcohols such as dodecanol, undecanol, octanol; triglycerides), disintegration promoters, disintegrants (wick agents, e.g. aerosil);

wetting agents; sweetening and flavouring agents (e.g. peppermint, mint, menthol, camphor); antioxidants; preservatives (e.g. sorbic acid and its salts, vitamins A and E), pH regulators;

permeation-promoting and absorption-promoting substances (e.g. saturated or unsaturated fatty acids; fatty acid esters, especially esters with methanol, ethanol and isopropanol, e.g. oleic acid ethyl ester, oleic acid methyl ester, lauric acid ethyl ester, lauric acid methyl ester, adipic acid methyl ester, adipic acid ethyl ester; fatty alcohols and their esters, especially esters; with acetic or lactic acid, e.g. ethyl oleate, ethyl laurate, ethyl palmitate; polyhydric aliphatic alcohols such as propanediol, or polyethylene glycols; sorbic fatty acid esters and their derivatives obtained by ethoxylation; fatty alcohol ethoxylates, polyoxyethylene fatty acid esters, lauric acid diethanolamide; oleic acid diethanolamide; tocopherol; lauric acid hexyl ester; 2-octyl dodecanol, dexpanthenol, isopropylidene glycerol, transcutol, DEET, solketal; menthol and other ethereal oils or components of such oils; as well as combinations thereof).

The above-mentioned auxiliary substances may preferably be contained in a total concentration of up to 50%-wt, especially in a total concentration of 1 to 15%-wt, each relative to the active substance-containing layer(s). By changing the type and quantity of the auxiliaries added, it is possible to influence the chemical or physical properties of the wafer, such as flexibility, mucoadhesive properties, disintegration capability, swelling capability, diffusion properties.

The invention further encompasses the use of at least one cholinergic active substance acting on the central nervous system, which is selected from the group comprising galanthamine, pharmaceutically acceptable salts of galanthamine, galanthamine derivatives and their pharmaceutically acceptable salts, for the production of film-forming buccal medicaments intended for transmucosal administration of the said active substance(s) for treating diseases or symptoms associated with, or caused by, a lack of acetylcholine-induced conduction and/or a disturbed regulation of neuronal nicotinic receptors.

Furthermore, the present invention comprises the use of film-shaped buccal medicaments containing at least one cholinergic active substance acting on the central nervous system, selected from the group comprising galanthamine, pharmaceutically acceptable salts of galanthamine, galanthamine derivatives and their pharmaceutically acceptable salts, for transmucosal administration of the said active substance(s) for treating diseases or symptoms associated with, or caused by, lack of acetylcholine-induced conduction and/or disturbed regulation of neuronal nicotinic receptors.

The film-shaped preparations according to the invention are, in particular, suitable for the medicament therapy of the following diseases and symptoms:

Alzheimer's disease (in all its manifestations and stages), especially impaired memory associated therewith (Alzheimer's dementia); in addition, Down syndrome, late stages of Down syndrome (especially dementia, loss of cognitive abilities); impaired memory having other causes.

Neurological diseases or symptoms, especially paralytic states in cases, or as a consequence of: poliomyelitis, myasthenia gravis, brain and spinal cord injuries, multiple sclerosis, amyotrophic lateral sclerosis, apoplexy, cranio-cerebral trauma, tumour diseases.

Chronic fatigue syndrome, schizophrenia; mania; disturbed sleep, especially snoring and apnoea; the effects of jetlag as well as other disorders of the physiological rhythm of body functions;

Disorders of the central nervous system caused by action of psychotropic substances, especially intoxications with such substances; poisoning with neurotoxins or chemical warfare agents (especially organophosphoric substances); disorders of the central nervous system occurring as a result of the action of psychotropic substances as a consequence of occasional or chronic use or abuse of addictive substances, narcotics or medicaments, or as side effects of use as intended of medicaments, especially repeated or prolonged use of medicaments, or as a consequence of acute poisoning, or as a consequence of chronic action of poisonous substances; especially impaired memory, as well as impairment of memory performance, impaired perception, impaired coordination of movements;

alcoholism or nicotine dependence, abuse of other chemical substances; especially treatment to reduce the craving for alcohol or to reduce the craving for nicotine. For these treatment purposes, it is also possible to administer galanthamine (or a derivative thereof)—preferably in a rapid-releasing, film-shaped buccal dosage form—in combination with further therapeutic active agents counteracting the respective abuse, in their respective suitable dosage forms, for example in combination with opiate antagonists (as in DE-A-101 34 038) for treating nicotine abuse, or in combination with substances having antiexcitatory action for treating alcohol abuse (as in DE-A-101 29 265). The said further active substances may also be contained in combination with galanthamine (or a derivative thereof) in a wafer according to the present invention.

The inventive wafers may furthermore be used for antidote treatment in cases of neuroleptic analgesia. Furthermore, the wafers according to the invention may also be used for further therapeutic treatment purposes not expressly mentioned herein.

The invention in addition encompasses methods of treatment of persons suffering from one of the above-mentioned diseases or from one the above-mentioned symptoms or who for other reasons require treatment with a cholinergic agent acting on the central nervous system. To this end, the person to be treated is buccally administered a therapeutically effective dose of at least one cholinergic agent acting on the central nervous system from the group comprising galanthamine, pharmaceutically acceptable salts of galanthamine, galanthamine derivatives and their pharmaceutically acceptable salts, in the form of film-shaped medicaments, as described above.

Administration is accomplished by introducing the film-shaped preparation into the oral cavity (buccal, sublingual administration) and, in the case of mucoadhesive films, by sticking the preparation to the buccal or gingival mucosa or other regions of the oral mucosa (e.g. palate or sublingual).

Depending on the active substance content, the release rate, the disintegration properties and the individually required doses, application is repeated in intervals of preferably 2 to 24 h, especially 6 to 12 h. The administered daily dose of galanthamine (and/or galanthamine derivative(s)) is between 10 and 750 mg, preferably 50 to 500 mg, depending on the body weight of the person and other factors.

1. Film-shaped medicament for buccal administration of galanthamine or a salt or derivative thereof, said medicament comprising at least one layer which contains a cholinergic active substance acting on the central nervous system, or a combination of at least two such active substances, said active substance(s) being selected from the group consisting of galanthamine, pharmaceutically acceptable salts of galanthamine, galanthamine derivatives and their pharmaceutically acceptable salts, and said film-shaped medicament being soluble in aqueous media and/or rapidly disintegrating in aqueous media, but not being mucoadhesive.

2. Film-shaped medicament according to claim 1, characterized in that the said layer or at least one of the layers has a polymer matrix serving as active substance reservoir, the polymer content amounting to 5 to 95%, preferably 15 to 75%-wt, with particular preference 20 to 50%-wt.

3. Film-shaped medicament according to claim 1, characterized in that within 30 min, preferably within 15 min, with particular preference within 5 min after application it releases such an amount of the active substance(s) contained therein to the oral cavity that an effective plasma level is achieved.

4. (canceled)

5. (canceled)

6. (canceled)

7. Film-shaped medicament according to claim 1, characterized in that it is of a bilayer or multilayer structure, with at least one layer containing active substance.

8. Film-shaped medicament according to claim 1, characterized in that at least one of the layers has a retarded active substance release.

9. Film-shaped medicament according to claim 1, characterized in that the active substance content is 0.1 to 30%-wt, preferably 1 to 20%-wt., each relative to the active substance-containing layer(s).

10. Film-shaped medicament according to claim 1, characterized in that the medicament contains galanthamine, or a salt or derivative of galanthamine, in combination with at least one further pharmaceutically active substance, preferably selected from the group of the acetylcholinesterase inhibitors.

11. Film-shaped medicament according to claim 1, characterized in that its layer thickness is 0.01 to 5 mm, preferably 0.03 to 2 mm, with particular preference 0.05 to 1 mm.

12. Film-shaped medicament according to claim 1, characterized in that it contains one or more auxiliaries selected from the group consisting of fillers, colourants, emulsifiers, plasticizers, disintegration promoters, disintegrants (wick agents), wetting agents, sweetening and flavouring agents, preservatives, pH regulators, permeation-enhancing substances and antioxidants.

13. Use of at least one cholinergic active agent acting on the central nervous system, selected from the group consisting of galanthamine, pharmaceutically acceptable salts of galanthamine, galanthamine derivatives and their pharmaceutically acceptable salts, for the production of a film-shaped buccal medicament intended for transmucosal administration of the said active substance(s) for treating diseases or disease symptoms accompanied by, or caused by, a lack of acetylcholine-induced conduction and/or a disturbed regulation of neuronal nicotinic receptors.

14. Use according to claim 13, characterized in that the said film-shaped medicament is a medicament comprising at least one layer which contains said cholinergic active agent or a combination of at least two such active agents and said film-shaped medicament being soluble in aqueous media and/or rapidly disintegrating in aqueous media, but not being mucoadhesive.

15. Use according to claim 13, characterized in that the said disease is Alzheimer's disease or that the said symptom is impaired memory occurring in the course of Alzheimer's disease.

16. Use according to claim 13, characterized in that the said treatment is the therapy of alcohol abuse, especially a treatment for reducing the craving for alcohol, or the therapy of nicotine abuse, especially a treatment for reducing the craving for nicotine.

17. Use according to claim 13, characterized in that the said treatment is an antidote treatment following neuroleptic anaesthesia.

18. Use according to claim 13, characterized in that the said treatment is a therapy of abuse of chemical substances or of the dependence on such substances, especially a therapy of intoxication with psychotropic substances.

19. Use according to claim 13, characterized in that the said symptoms or diseases are symptoms of jet lag or other disorders of the physiological rhythm of body functions.

20. Use according to claim 13, characterized in that the said symptoms or diseases are chronic fatigue syndrome or disturbed sleep.

21. Use according to claim 13, characterized in that the said disease is schizophrenia or a mania.

22. Use according to claim 13, characterized in that the said diseases or symptoms are neurological illnesses and symptoms, especially paralytic symptoms.

23. Use according to claim 13, characterized in that the medicament is used for the treatment of disorders of the central nervous system occurring as a consequence of the action of psychotropic substances caused by occasional or chronic use or abuse of addictive substances, narcotics or medicaments, or as a side effect of the use of medicaments as intended, especially repeated or prolonged use of medicaments, or as a consequence of acute poisoning, or as a consequence of the chronic action of poisons, in humans or other vertebrates.

24. Use according to claim 23, characterized in that the said symptoms are symptoms from the group consisting of cognitive disorders, especially impaired memory, as well as impairment of memory performance, impaired perception, impaired coordination of movements.

25. Method for treating a person suffering from one of the diseases or symptoms mentioned in claim 13, or who is affected by a dependence on one or more chemical substances, in which method the person to be treated is buccally administered a therapeutically effective dose of at least one cholinergic active substance acting on the central nervous system from the group comprising galanthamine, pharmaceutically acceptable salts of galanthamine, galanthamine derivatives and their pharmaceutically acceptable salts, in the form of a film-shaped medicament.

26. Method according to claim 25, characterized in that the film-shaped medicament is a medicament comprising at least one layer which contains said cholinergic active agent or a combination of at least two such active agents and said film-shaped medicament being soluble in aqueous media and/or rapidly disintegrating in aqueous media, but not being mucoadhesive.
